(19) United States
(12) Patent Application Publication
(10) Pub. No.: US 2005/0074789 A1
(43) Pub. Date: Apr. 7, 2005

(54) Methods and devices for isolating single polymeric molecules

(76) Inventors: Xing Su, Cupertino, CA (US); Narayan Sundararajan, San Francisco, CA (US)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/748,802

(22) Filed: Dec. 30, 2003

Related U.S. Application Data
(60) Provisional application No. 60/509,707, filed on Oct. 7, 2003.

(51) Int. Cl. C12Q 1/68; G01N 33/53; G01N 33/542

(52) U.S. Cl. 435/6; 435/7.9

(57) ABSTRACT

Methods and devices for immobilizing single polymeric molecules are disclosed. A method for isolating a single polymeric molecule comprises contacting polymeric molecules with agents under conditions that permit formation of agent-polymeric molecule complexes, said polymeric molecules immobilized at positions on a substrate separated by at least about two times the length of the polymeric molecules, said agents comprising a binding partner having binding affinity for a label of the polymeric molecules, and releasing at least one of the agent-polymeric molecule complexes from the substrate to isolate a single polymeric molecule.

### CROSS-REFERENCE TO RELATED APPLICATION

This claims the benefit under 35 U.S.C. § 119(e) of U.S. provisional patent application Ser. No. 60/509,707 filed Oct. 7, 2003, the disclosure of which is incorporated herein by reference.

### TECHNICAL FIELD

The disclosure is generally related to methods and devices for immobilizing single polymeric molecules. More specifically, methods and devices for isolating, manipulating, and controllably dispensing individual polymeric molecules are disclosed.

### BACKGROUND

Single molecule analysis is of interest for a variety of reasons, including its potential for providing high-resolution information for individual genotypes. Such information may be used, for example, to identify genetic variations that cause or contribute to disease states and/or to increase pharmaceutical efficacy (Braslavsky et al., P.N.A.S., 100: 3960-3964 (2003)). Generating the desired information, however, essentially requires that a single targeted DNA molecule be physically isolated from a bulk sample and manipulated in a manner that permits subsequent analysis.

Polymeric molecules, such as DNAs, having various functional groups attached to their ends have been attached to solid supports by covalent bonds formed between the attached functional groups and complementary groups present on the solid support surface. For example, single DNA molecules covalently attached to beads have been manipulated with optical tweezers (T. Perkins et al., Science, 264: 822-826 (1994)). A significant disadvantage of such techniques involves limiting the number of polymeric molecules attached to the beads or other solid support surfaces.

Polymeric molecules have been hybridized to complementary molecules covalently attached to a substrate, and subsequently released from the substrate. For example, infrared laser irradiation has been used to thermally denature and release DNA molecules immobilized to specific areas of a conventional DNA chip (K. Okano et al., Sensors and Actuators, 64:88-94 (2000)). The diameter of the focused laser beam may, however, cause more than a single polymeric molecule to thermally denature and thus be released from the substrate.

### BRIEF DESCRIPTION OF THE DRAWINGS

Exemplary aspects and features of the methods and devices in accordance with the disclosure are described and explained in greater detail below with the aid of the drawing figures in which:

FIG. 1 illustrates a microfluidic device in accordance with one embodiment of the invention and includes an expanded, projected view of an exemplary microfluidic device substrate;

FIG. 2 is a cross-sectional view along line 2-2 of the microfluidic device depicted in FIG. 1; and,

FIG. 3 illustrates a microfluidic device in accordance with an additional embodiment of the invention.

### DETAILED DESCRIPTION

The disclosure provides methods and devices for efficiently immobilizing and isolating single molecules. The disclosed methods have been exemplified using the microfluidic devices described herein.

One embodiment according to the invention provides methods for immobilizing and isolating single polymeric molecules (e.g., DNA molecules) on a substrate, and controllably releasing the single polymeric molecules into solution, thereby making it possible to isolate and selectively dispense single molecules for subsequent single molecule analysis.

A method for isolating a single polymeric molecule in accordance with one embodiment of the invention includes contacting polymeric molecules that are immobilized at binding positions on a substrate with agents, under conditions that permit formation of agent-polymeric molecule complexes, and releasing one of the agent-polymeric molecule complexes from the substrate to isolate a single polymeric molecule. Typically, the agent comprises a binding partner having binding affinity for a label of the polymeric molecules and the substrate binding positions are separated by a distance on the substrate such that the agent is capable of forming a complex with only one immobilized polymeric molecule. In one aspect, the binding positions are separated by a distance on the substrate of at least about two times the length of the polymeric molecules.

The methods can further include washing the substrate (i.e., the surface to which the polymeric molecules are immobilized, but not necessarily the support surface). The substrate can be selected from the group consisting of gold substrates, aluminum substrates, glass substrates, silicon substrates, and polymeric substrates such as poly(methyl methacrylate) and poly(dimethyl siloxane). Furthermore, the substrate can be any metal layer or any organic polymer layer that can be modified to provide binding positions.

The polymeric molecule can include a first oligonucleotide, and the binding position can include a second oligonucleotide, which is complementary to at least a portion of the first oligonucleotide. Immobilizing can comprise hybridization of the first oligonucleotide to the second oligonucleotide. The terms oligomer and oligo are used interchangeably throughout the disclosure. According to one aspect in accordance with the disclosure, an oligonucleotide is an exemplary oligomer.

Releasing can be effected by heating, adding a pH adjusting compound to the system, changing the salt concentration of the system or otherwise disrupting the hydrogen bonds formed between base pairs, for example by adding a disrupting agent such as guanidine salts, urea, dimethyl sulfoxide (DMSO), and/or formamide, which is capable of disrupting hydrogen bonds formed between base pairs. The heating temperature, pH change, salt concentration, and disrupting agent concentration will vary depending on the melting temperature of the hybridized first nucleotide/second nucleotide complex, but can be easily determined by one of ordinary skill in the art. For example, experimental results demonstrate that the heating temperature of single molecule release correlates well with the expected theoretical melting temperature of the hybridized base pairs formed between the first oligonucleotide and the second oligonucleotide. Additionally, a restriction enzyme could be used if a portion of the polymeric molecule which is hybridized to the binding site oligomer on the substrate includes a restriction enzyme site.

The methods can further include transporting the at least one agent-polymeric molecule complex. According to one aspect, for example, an agent-polymeric molecule complex can be transported to a desired location using optical tweezers. According to another aspect, an agent-polymeric molecule complex can be transported by the flow of fluid through the device.

The polymeric molecules (e.g., nucleic acid molecules) are preferably modified/labeled on one end and immobilized to specific binding positions on a substrate surface where the shortest distance between two adjacent binding positions is at least about two times the length of the target polymeric molecules. A labeled polymeric molecule preferably binds to the substrate surface via the non-labeled end only. Unbound polymeric molecules (i.e., polymeric molecules that have not attached to a binding position) are typically washed off of the substrate surface after the immobilization of the polymeric molecules.

The polymeric molecules are subsequently contacted with agents (e.g., tags, carriers, and other such components known in the art) which can be manipulated individually and have specific binding sites for a label of the target polymeric molecules which are immobilized at binding positions on the substrate. Free agents (i.e., agents that have not been attached to a polymeric molecule) can be washed from the substrate surface. Exemplary agents include surface-functionalized microsphere beads. Suitable beads of varying sizes are commercially available (Bangs Laboratories, Inc., Fishers, Ind.). The microsphere beads typically have a diameter between about 0.1 microns (μ) and about 20μ, preferably between about 0.5μ and about 10μ, and most preferably between about 1μ and about 5μ. The microsphere beads typically comprise materials such as polystyrene, glass, polysaccharides such as agarose, and latexes such as styrene butadiene.

Video microscopy experiments have confirmed the immobilization of a single polymeric molecule to the substrate. For example, such video microscopy experiments have shown an agent, such as a microsphere bead, exhibiting Brownian motion within a confined location, thereby indicating the presence of a single polymeric molecule attached to the substrate. By detaching the polymeric molecule at the end attached to the substrate surface, a single molecule can be isolated by moving or transporting the agent, as previously described. Video microscopy experiments have further demonstrated the controlled release of the polymeric molecules from the substrate surface. Conventional microscopic techniques such as dark field microscopy, bright field microscopy, differential interference contrast microscopy, and fluorescent microscopy methods can also be used to demonstrate polymeric molecule immobilization and the controlled release of the polymeric molecules from the substrate surface.

Prior to immobilizing polymeric molecules thereto, the substrate typically is modified to include binding positions. For example, a suitable glass substrate can be treated with sodium hydroxide to expose reactive, hydroxyl groups. The hydroxyl groups of the substrate can be further reacted with an aldehyde-containing silane reagent to form an aldehyde-activated substrate. Aldehyde-activated substrates are commercially available (NoAb BioDiscoveries Inc., Ontario, Canada). Suitable substrates can alternatively be reacted with silane reagents containing carboxyl groups, amino groups, and/or epoxy groups to form carboxyl-activated substrates, amino-activated substrates, and/or epoxy activated substrates.

The activated substrate can then be treated with a mixture comprising a receptor and a blocking agent. For example, according to one exemplified embodiment of the invention, an aldehyde-activated substrate is reacted with a mixture comprising a receptor and bovine serum albumin (BSA). The aldehyde functional groups of the substrate react with amines present on the proteinaceous receptors and blocking agents to form covalent bonds, thereby attaching the receptors and the blocking agents to the substrate. The attached receptors provide individual and delocalized precursor binding positions on the substrate.

The precursor binding positions of the substrates can be further modified by immobilizing oligomeric or polymeric molecules to their surface to provide binding positions. For example, an oligonucleotide having a labeled 5′ end can be reacted with the receptors attached to the substrate. Preferably, the receptor has binding affinity for the oligonucleotide label. In one representative embodiment of the invention, the oligonucleotide label is biotin and the receptor attached to the substrate is avidin, and the oligonucleotide is immobilized at the precursor binding site position via the biotin moiety to form binding positions. In another representative embodiment of the invention, the oligonucleotide label is an antigen and the receptor attached to the substrate is an antibody for the antigen, and the oligonucleotide is immobilized at the precursor binding site position via the antigen to form binding positions. For example, the antigen can be digoxigenin and the antibody can be anti-digoxigenin antibody; the antigen can be fluorescein and the antibody can be anti-fluorescein antibody; and, the antigen can be cholesterol and the antibody can be anti-cholesterol antibody.

Substrate binding positions separated by at least about two times the length of the polymeric molecules can be provided by treating the activated substrate with a mixture comprising a receptor and a blocking agent, the mixture having a ratio of receptor to blocking agent of about one to about ten. The mixture ratio can also be about one receptor to about 100 blocking agents, and about one receptor to about 1000 blocking agents. Alternatively, the ratio can also be about one receptor to about 10,000 blocking agents, one receptor to about 100,000 blocking agents, one receptor to about 1,000,000 blocking agents, and about one receptor to about 10,000,000 blocking agents. Other suitable ratios can be determined by one having ordinary skill in the art. Substrate binding positions separated by at least about two times the length of the polymeric molecules can be provided by treating the activated substrate first with a solution containing the receptor and subsequently with a solution comprising the blocking agent. Binding positions separated by at least two times the length of the polymeric molecules are measured by the final effect achieved by the previously described surface treatment procedures, which are governed but not measured by the molecular ratios provided herein.

To minimize non specific binding of the beads to the substrate, a solution containing a receptor, such as avidin, can be mixed in a 0.5 weight percent (wt. %) BSA solution. The device should subsequently be washed with a 0.5 wt. % BSA solution. The concentration range for the receptor containing solution, e.g., a solution containing avidin, should be between about 0.01 nanomolar (nM) and about 10 nM. Approximately one to two milliliters (ml) of a receptor containing solution should be sufficient to provide precursor binding sites in a microfluidic device according to one embodiment of the invention (e.g., a microfluidic device having a width of about 50 microns, and a length of about five centimeters).

Alternatively, a suitable substrate can be modified to include binding positions separated by at least about two times the length of the polymeric molecules by treating the substrate surface with a mixture comprising a functionalized oligomer and a blocking agent. For example, a gold substrate can be treated with mixture including a thiol-modified nucleic acid oligomer (functionalized oligomer) and hexadecanethiol (blocking agent). The mixture ratio of functionalized oligomer to blocking agent can be about one to about ten. Alternatively, the ratio can also be about one functionalized oligomer to about 10,000 blocking agents, one functionalized oligomer to about 100,000 blocking agents, one functionalized oligomer to about 1,000,000 blocking agents, and about one functionalized oligomer to about 10,000,000 blocking agents. Again, other suitable ratios can be determined by one having ordinary skill in the art. Substrate binding positions separated by at least about two times the length of the polymeric molecules can be also provided by treating the activated substrate first with a solution containing the functionalized oligomer and subsequently with a solution comprising the blocking agent.

The attached oligomers (e.g., oligonucleotides) of the binding positions are subsequently contacted with ‘target’ polymeric molecules. Target, labeled polymeric molecules that are complementary to the attached oligomers can hybridize to the attached oligomers, and are immobilized to the target polymeric molecules at the binding positions on the substrate. According to one embodiment of the invention, the oligomer is synthesized to be complementary to a specific (i.e., known) region in a target polymeric molecule. Alternatively, the oligomer can be synthesized to be complementary to a specific region in a target molecule, which has been added or ligated to the target molecule.

Target molecules can be prepared by digesting a DNA sample with two different restriction enzymes to create DNA fragments with two different ends. In one aspect, a “hairpin-like oligonucleotide” containing a biotin moiety in the middle and a restriction enzyme site or at least an appropriate overhanging end at its end is ligated to one desired end of the digested DNA, and an oligonucleotide, which is designed to be complementary to the binding site oligomer on the substrate, can be added to the other end. Alternatively, the digested DNA sample can be treated with a polymerase to provide a tail (whose sequence is known by virtue of controlled polymerization, e.g., poly-dT-tail) for potential hybridization to the substrate binding site oligomer.

Because the free terminus of the polymeric molecules is labeled, contacting the polymeric molecules with an agent comprising a binding partner having binding affinity for the label permits the formation of agent-polymeric molecule complexes. Single molecule release of the ‘target’ polymeric molecules to isolate the individual molecule can be achieved as previously described.

According to an additional embodiment of the invention, a method for isolating a single polymeric molecule includes providing an agent-polymeric molecule complex having only one bound polymeric molecule, immobilizing the agent-polymeric molecule complex to a substrate at a position having a binding position that interacts with at least a portion of the polymeric molecule, and releasing the agent-polymeric molecule complex to isolate a single polymeric molecule. The methods can further include removing the polymeric molecules on the agent-polymeric molecule complex that are not immobilized to the substrate.

The methods can further include washing the substrate (i.e., the surface to which the polymeric molecules are immobilized, but not necessarily the support surface). The substrate can be selected from the group consisting of gold substrates, aluminum substrates, glass substrates, silicon substrates, and polymeric substrates such as poly(methyl methacrylate) and poly(dimethyl siloxane). Furthermore, the substrate can be any metal layer or any organic polymer layer that can be modified to provide binding positions.

The polymeric molecule can include a first oligonucleotide, and the binding position can include a second oligonucleotide, which is complementary to at least a portion of the first oligonucleotide. Immobilizing can comprise hybridization of the first oligonucleotide to the second oligonucleotide.

Releasing can be effected by heating, adding a pH adjusting compound to the system, changing the salt concentration of the system or otherwise disrupting the hydrogen bonds formed between base pairs, for example by adding a disrupting agent such as guanidine salts, urea, dimethyl sulfoxide, and/or formamide, which is capable of disrupting hydrogen bonds formed between base pairs. The heating temperature, pH change, salt concentration, and disrupting agent concentration will vary depending on the melting temperature of the hybridized first nucleotide/second nucleotide complex, but can be easily determined by one of ordinary skill in the art. Additionally, a restriction enzyme could be used if a portion of the polymeric molecule which is hybridized to the binding site oligomer on the substrate includes a restriction enzyme site.

The methods can further include transporting the at least one agent-polymeric molecule complex. According to one aspect, for example, an agent-polymeric molecule complex can be transported to a desired location using optical tweezers. According to another aspect, an agent-polymeric molecule complex can be transported by the flow of the fluid through the device.

According to the methods in accordance with this embodiment, polymeric molecules (e.g., nucleic acid molecules) are modified/labeled on both ends, e.g., on or near either the 5 ends or the 3′ ends, but not both the 5′ ends and the 3′ ends. In an exemplary embodiment, a double stranded DNA molecule (dsDNA) is ligated, for example, with a biotin-labeled double stranded linker, known in the art and commercially available, and subsequently denatured to provide two single stranded, end-labeled DNA molecules that are labeled on the 5′ or 3′ ends, but not both. Biotin-labeled dsDNA (labeled at either the 5′ ends or the 3′ ends, but not both the 5′ ends and the 3′ ends) can also be generated prior to ligation to include a biotin label on only one strand of the DNA, using for example, fill-in of overhanging ends in those instances where a single restriction enzyme has been used to digest the nucleic acid (thereby producing identical overhanging ends at the end terminus of the nucleic acid). Additionally, biotin-labeled dsDNA can be produced by ligating ‘linker’ DNA molecules, using a polymerase and biotin-labeled nucleotides, and carrying out fill-in reactions as described above.

In some cases, the same linker DNA molecule can be ligated to both the 5′ and 3′ ends that have been generated from the restriction enzyme digestion. To create different modified ends of the DNA (partial single-stranded DNA termini), two different linkers with the same ligation sites can be used to ligate to the 5′ and 3′ ends generated by the same restriction enzyme. If different 5′ and 3′ ends are desired, two different restriction enzymes are used, and the DNA fragments are isolated based on size prediction, according to known information.

In an alternative embodiment, the obtained single-stranded DNAs are labeled at both the 3′ (by polymerization or terminal transfer) and the 5′ ends (by ligation). The 3′ and 5′ ends can be labeled differently, for example one end with digoxigenin and the other end with biotin.

The single stranded, end-labeled DNA molecules can be mixed with an agent, such as microsphere beads, under conditions that permit formation of agent-polymeric molecule complexes. The DNA molecules may be provided in excess, i.e., such that there is more than one DNA molecule per bead. In an exemplary embodiment, the DNA molecules are labeled and the beads have a coating comprising a binding partner having binding affinity for the label. For example, the DNA molecules can be labeled with biotin and the agents can be microsphere beads coated with streptavidin (or alternatively, with avidin). In another representative embodiment of the invention, the DNA label is an antigen and the binding partner is an antibody for the antigen. For example, the antigen is digoxigenin and the antibody is anti-digoxigenin antibody; the antigen is fluorescein and the antibody is anti-fluorescein antibody; and, the antigen can be cholesterol and the antibody can be anti-cholesterol antibody.

Substrates can be functionalized by immobilizing oligomeric or polymeric molecules to their surface, as described above. The immobilized oligomeric molecules can be contacted with a solution containing the agent-polymeric molecule complexes. For example, according to one embodiment of the invention, the solution containing the agent-polymeric molecule complexes is flowed over the oligomer-modified substrate surface. Of course, the solutions can also be provided under static conditions. When the target single stranded DNA is labeled at the 5′ end, the immobilized oligomer should be complementary to the 3′ end of the target, and when the target single stranded DNA is labeled at the 3′ end, the immobilized oligomer should be complementary to the 5′ end of the target. Contacting the immobilized oligomeric molecules with a solution containing the agent-polymeric molecule complexes results in the hybridization of an immobilized oligomer to at least a portion of a polymeric molecule bound to an agent, thereby attaching the agent-polymeric molecule complex to the substrate surface.

The attached agent-polymeric molecule complex can be contacted with an enzyme solution to selectively deconstruct the single stranded DNA molecules on the surface of the agent, without affecting the DNA molecule hybridized to the immobilized oligomer. For example, according to one embodiment of the invention, the enzyme solution is flowed over the oligomer-modified substrate surface. The exonuclease can be 5′ specific if the 3′ end is labeled (and vice versa). Single molecule release of the ‘target’ polymeric molecules can be achieved as previously described.

According to another embodiment of the invention, a method for isolating a single polymeric molecule includes introducing a mixture comprising agent-polymeric molecule complexes having varying numbers of bound polymeric molecules into an applied electric field, and separating the agent-polymeric molecule complexes having only one bound polymeric molecule from the mixture based on mobility to isolate a single polymeric molecule. Separation of the mixture occurs because polymeric molecule (e.g., nucleic acid) attachment to an agent changes the charge of the formed agent-polymeric molecule complex, therefore also affecting its mobility in an applied electrical field.

The methods may further include determining the mobility of an agent-polymeric molecule complex having only one bound polymeric molecule under the applied electric field. For example, the mobility of agents having no bound polymeric molecules can be easily measured. The ratio of polymeric molecules to carriers can be varied and the mobility distribution of the agent-polymeric molecule complexes can be determined. Based on these data, the mobility of carrier with a single bound polymeric molecule can be predicted.

In the methods according to one embodiment of the invention, the agents can comprise microsphere beads having a coating comprising a binding partner having binding affinity for a label of the polymeric molecule. The label can be biotin and the binding partner can be either avidin or streptavidin. Alternatively, the label can be an antigen and the binding partner can be an antibody for the antigen, as previously described herein. For example, the label can be digoxigenin and the binding partner can be anti-digoxigenin antibody; the label can be fluorescein and the binding partner can be anti-fluorescein antibody; and, the label can be cholesterol and the binding partner can be anti-cholesterol antibody.

The polymeric molecule can be a nucleic acid, including double stranded nucleic acid, deoxyribonucleic acid, and ribonucleic acid.

According to an additional embodiment of the invention, a microfluidic device includes a micromold comprising a chemically inert material and having a top surface, a bottom surface, a sample inlet, a sample outlet, and a microchannel pathway defined between the sample inlet and the sample outlet, a substrate adhered to the bottom surface, the substrate having binding positions for immobilizing polymeric molecules, said binding positions separated by at least about two times the length of the polymeric molecules, and a heating element adapted to heat the substrate.

The micromold can comprise a silicone material. Typically, the microchannel has a width between about 10 microns and about 200 microns, and a length between about 0.25 centimeters and about five centimeters.

The binding positions can comprise a polymeric molecule. The substrate binding positions can be provided as described above. The polymeric molecule can comprise a thiol-modified oligonucleotide or a labeled oligonucleotide. The heating element can comprise a thin-film resistive heater. In one embodiment of the invention, the heating element is the substrate.

The microfluidic device can also further include a passivation layer between the substrate and the heating element. When the microfluidic device includes a passivation layer, a first pattern formed by the resistive heater can be different from a second pattern formed by the substrate. When the first pattern formed by the resistive heater differs from the second pattern formed by the substrate, the microfluidic device provides individually addressable binding positions, thereby facilitating the controllable release of an individual polymeric molecule adhered to the individually addressable binding position of the substrate. According to one aspect, the first pattern and the second pattern intersect at discrete locations to provide such individually addressable binding positions. According to a preferred embodiment of this aspect, the first pattern and the second pattern intersect at an approximately 90° angle.

According to another embodiment of the invention, a microfluidic device includes a micromold comprising a chemically inert material and having a sample well, a first end, a second end, and a microchannel pathway defined between the first end and the second end, and a first electrode disposed proximate to the first end and a second electrode disposed proximate to the second end. The inner surface of the microfluidic device can be modified such that it is neutral, negative or positively charged.

The microfluidic device can further include a collection chamber having a third end and a fourth, collection end, the collection chamber being substantially transverse to the microchannel. A third electrode can be disposed proximate to the collection chamber third end and a fourth electrode can be disposed proximate to the collection chamber fourth, collection end. The microfluidic device can further include a switching circuit between the first electrode and the second electrode. Further, the microfluidic device can further include a power supply operatively connected to the switching circuit.

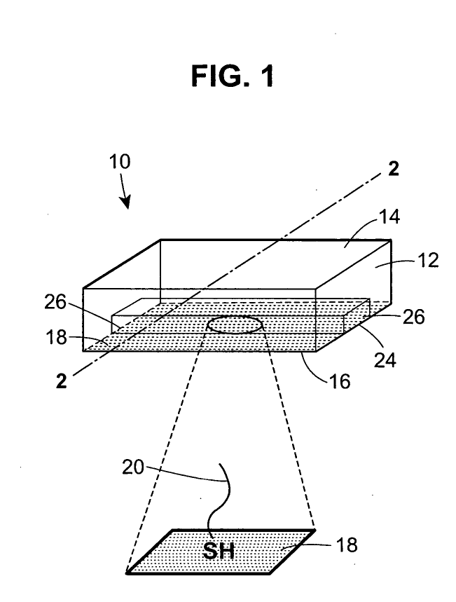

Referring now to the drawing figures, a microfluidic device in accordance with one embodiment of the invention is generally referred to by reference numeral 10. As illustrated in FIG. 1, microfluidic device 10 includes micromold 12. Microfluidic device has a top surface 14 and a bottom surface 16. Microfluidic device includes a substrate 18. Substrate 18 provides binding positions 20 (exemplified in the expanded, projected view as a thiol-modified oligomer immobilized to the substrate). Microfluidic device 10 further includes a sample inlet 22, a sample outlet 24, and a microchannel pathway 26 defined between the sample inlet and the sample outlet.

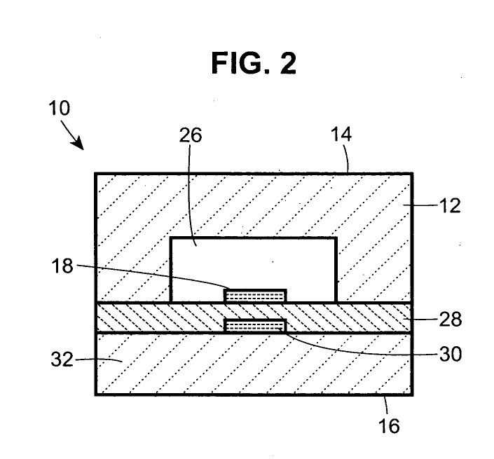

FIG. 2 shows a cross-sectional view of the microfluidic device 10 depicted in FIG. 1 along line 2-2. Microfluidic device 10 can include passivation layer 28. Further, microfluidic device 10 can include heating element 30 and supporting surface 32. Heating element 30 is depicted as a thin-film resistive heater. An electrical contact (not shown) is included to pass current through the heating element 30. A thermocouple (not shown) can be operatively connected to the heating element to measure the temperature at which a single molecule is released (heating temperature). A processing unit (not shown) can be used to program and control the heating temperatures. Additionally, other heating elements 30 can be used to release polymeric molecules immobilized to binding positions 20, including heating means such as a hot plate or a focused laser beam.

Passivation layer 28 serves to decouple substrate 18 from heating element 30. Passivation layer is often included to mitigate electrolysis problems that occur when substrate 18 is directly heated. Furthermore, controlled release of the immobilized polymeric molecules can be attained by decoupling substrate 18 and heating element 30, as is described in further detail below. Nonetheless, in one embodiment of the invention, the device 10 does not include a passivation layer 28, and the substrate 18 is also the heating element 30. Suitable passivation layers 28 include SU-8 photoresist, spin-on glass (SOG), plasma enhanced chemical vapor deposition (PECVD) silicone dioxide, and PECVD silicon oxynitride. Silicon oxide and silicon oxynitride layers are preferred and may be deposited by any conventional deposition technique, including chemical vapor deposition and thermal growth. The passivation layer 28 is typically at least about 1 micron thick. In an alternative embodiment, passivation layer 28 can be modified to provide binding sites, i.e., to be the substrate 18.

Techniques such as soft lithography and photolithography, which have been used in the semiconductor industry, can be used to fabricate micromold 12 of microfluidic device 10. For example, designs of micromold 12 were drawn to scale using CAD software. The designs were then printed onto transparencies using a high-resolution printer to form a transparency mask. “Photoresist on Silicon” masters for micromolding were prepared by standard photolithographic techniques using the transparency masks and a photoresist. These patterned masters were then silanized and used for micromolding with a silicone material such as poly(dimethyl siloxane) (PDMS). For example, PDMS precursor was poured onto the silanized master and then cured. The cured PDMS containing the channel structure was then bonded to the supporting surface 32 by applying pressure to enclose the channels. Typically, the microchannel pathways 26 were approximately 100 microns in width and between about two centimeters and about three centimeters in length.

The substrate 18 can also be prepared using standard lithographic techniques. For example, a photoresist can be deposited on substrate support surface 32 and exposed through a mask. The exposed photoresist can be developed. A suitable heating element 30 or substrate 18 material can be deposited by, for example, sputter deposition. In one embodiment, a thin layer of titanium or chromium having a thickness of about 80 Å is deposited, followed by subsequent deposition of a thin layer of gold having a thickness of about 240 Å. The photoresist is then lifted off of substrate support surface 32, thereby providing a substrate 18 and/or heating element 30 on the substrate support surface 32.

If a passivation layer 28 is to be incorporated into device 10, the initial structure formed on the substrate support surface 32 is a heating element 30, and a suitable passivation material can be deposited over the heating element 30 and over the substrate support surface 32 to form a passivation layer 28. Subsequently, a photoresist can be deposited on passivation layer 28 and exposed through a mask. The exposed photoresist can be developed. A suitable substrate 18 material can be deposited by, for example, sputter deposition. In one embodiment, a thin layer of titanium is deposited, followed by subsequent deposition of a thin layer of gold, as provided above. The photoresist is then lifted off of passivation layer 28, thereby providing a substrate 18 on the passivation layer 28. FIG. 2 shows a structure incorporating such a passivation layer 28. While FIG. 2 shows a structure wherein the deposition pattern of the heating element 30 is the same as the deposition pattern of the substrate 18, the pattern formed by the heating element 30 can be different from the pattern formed by the substrate 18, to provide an additional way of locally heating and releasing molecules immobilized to the substrate 18.

According to this aspect, a molecule immobilized to a binding position on the substrate can be individually addressed and controllably dispensed from the substrate surface by virtue of the different heating element and substrate patterns. For example, current applied to the heating element will only release those molecules immobilized at binding positions on the substrate that intersect with the heating element.

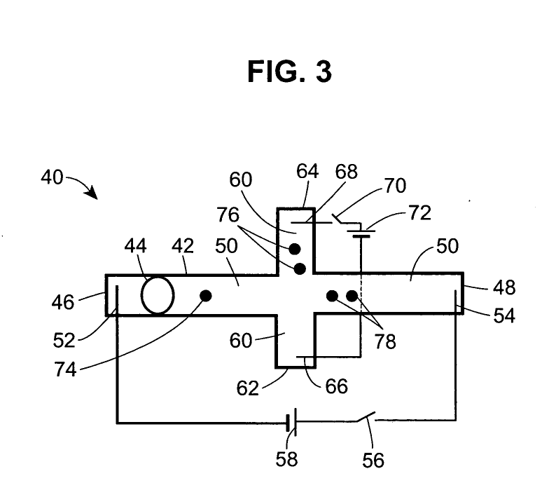

Referring now to FIG. 3, a microfluidic device in accordance with another embodiment of the invention is generally referred to by reference numeral 40. Microfluidic device 40 includes micromold 42. Micromold 42 includes a sample well 44, a first end 46, a second end 48, and a microchannel pathway 50 defined between the first end 46 and the second end 48. A first electrode 52 is disposed proximate to the first end 46 and a second electrode 54 is disposed proximate to the second end 48. A switching circuit 56 is located between the first electrode and the second electrode. Switching circuit 56 permits an applied field to be turned on and off. A power supply 58 is typically operatively connected to the switching circuit 56.

Microfluidic device 40 can include a collection chamber 60 having a third end 62 and a fourth, collection end 64. Typically, the collection chamber 60 is substantially transverse to the microchannel pathway 50. A third electrode 66 can be disposed proximate to the third end 62 and a fourth electrode 68 can be disposed proximate to the fourth, collection end 64. A switching circuit 70 is located between the third electrode 66 and the fourth electrode 68. Switching circuit 70 permits an additional field to be applied to the collection chamber, thereby facilitating separation of the desired polymeric-agent complexes. A power supply 72 is typically operatively connected to the switching circuit 70.

FIG. 3 further shows the application of a method in accordance with one embodiment of the invention. For example, FIG. 3 shows the separation of agent-polymeric molecule complexes having only one bound polymeric molecule from a mixture comprising agent-polymeric molecule complexes having varying numbers of bound polymeric molecules in an applied electric field. In FIG. 3, an agent having no bound polymeric molecules is depicted as reference number 74, two agent-polymeric molecule complexes having only one bound polymer are depicted as reference number 76, and two agent-polymeric molecule complexes having more than one bound polymer are depicted as reference number 78. An initial applied field between the first end 46 and the second end 48 results in an initial separation of the mixture. When the desired polymeric-agent complexes 76 (i.e., those agent-polymeric molecule complexes having only one bound polymeric molecule) have migrated to the collection chamber 60, switching circuit 56 can be turned off such that the field applied between the first end 46 and the second end 48 is no longer applied. Switching circuit 70 can then be turned on to promote movement of the desired polymeric-agent complexes 76 towards the collection chamber, collection end 64, to isolate the single polymeric molecule.

### EXAMPLES

The disclosed methods and devices for isolating a single polymeric molecule can be better understood in light of the following examples, which are merely intended to illustrate the disclosed methods and devices and are not meant to limit the scope in any way.

### Example 1

‘Target’ Polymeric Molecule Preparation

Modified λ-phage DNA (48.5 kbps) was used as the target DNA in this study. λ-phage DNA was modified through ligation using DNA oligomers such that one end of the DNA had a complementary sequence that hybridizes to a substrate binding site oligomer, and the other end had a biotin label for attachment to an agent (e.g., a polystyrene (PS) bead). After ligation, modified λ-DNA molecules were separated from the short DNA oligomers by adding polyethylene glycol to cause the precipitation of the modified λ-DNA molecules. Precipitated ‘target’ DNA was collected and dissolved in buffer and stored at 4° C. before use.

Specifically, for 200 microliters (μl) ligation reaction, 40 μl of stock solution of lamba-phage DNA (0.5 ng/μl), 10 μl of 10 μM LcosA30 (an exemplary oligomer to be ligated to the 5′ overhang of the lambda-DNA), 10 μl of 10 μM Rcos (an exemplary oligomer to be ligated to the 3′ overhang of the lambda-DNA), and 20 μl of 10× ligase buffer were mixed together gently (after adjusting the volume to 200 μl with water) and heated to 65° C. for 10 min. 4 μl of T4 DNA ligase was added to the ligation reaction mixture after the mixture was cooled down to approximately room temperature (˜25° C.). The ligation reaction mixture was then stored at room temperature for about 9 to about 15 hours such that the ligation reaction could proceed to completion.

In order to separate the short oligomers from the modified lambda-DNA, precipitation using poly(ethylene glycol) (PEG) was performed. According to this procedure, equal volumes of solutions containing 20 wt. % PEG and 2M NaCl were added to the modified lambda-DNA solution. The resulting solution was mixed gently until the modified lambda-DNA precipitates from solution. The supernatant solution was then removed by centrifugation and discarded. The DNA pellets were resuspended in 1×TE buffer (10 mM Tris HCl, pH 7.8 and 1 mM EDTA) to form a DNA solution. The remaining PEG and NaCl in the DNA solution were removed after adding ethanol to provide a 70% ethanol solution (by volume), thereby precipitating the modified lambda-DNA again. Finally, the DNA pellets were resuspended in 1×TE buffer. Each of the resultant modified lambda-DNA molecules is expected to have a single-stranded region at one end and a biotin label at the other end.

The following procedure was used to conduct static (no flow) experiments:

Substrate Modification

A 1 micromolar solution of thiol-modified DNA oligomer (Qiagen-Operon, Valencia, Calif.) was pipetted onto the surface of a gold thin film substrate and incubated at room temperature for 3-4 hrs. The surface was then washed with phosphate buffer saline (1×PBS) several times to remove unbound oligomers.

Immobilization of Target DNA

A 10 nanomolar solution of ‘target’ DNA dissolved in 1×PBS was pipetted onto the substrate and incubated at room temperature for 1-2 hrs. After immobilization (here, by hybridization), the substrate was then rinsed with 1×PBS more than three times to remove unhybridized DNA molecules.

Formation of Agent-Polymeric Molecule Complexes

After immobilization, a solution of streptavidin-coated polystyrene (PS) beads (1 μm diameter; 1:10 dilution of original solution in PBS obtained from Polysciences, Inc.) was incubated on the substrate for 1 hr to attach the beads to the biotinylated end of the ‘target’ DNA. The beads allowed the molecules to be visualized, and served as handles for optical manipulation after release of the polymeric molecule from the substrate.

For the dynamic (within microfluidic channels) experiments:

The reagents were pumped through the channels in the same order as in the static case, for 5 min by applying vacuum, followed by incubation within the microfluidic channels for time periods comparable to those used in the static experiments.

Single Molecule Isolation in Static Conditions

The density of single ‘target’ DNA molecules hybridized using static conditions was 3-5 molecules per 100 μm×100 μm square area. The beads attached to ‘target’ DNA exhibited Brownian motion but were restrained to within a radius of about two to three microns. DNA immobilization and bead attachment were further confirmed by using a standard upright optical microscope equipped with optical tweezers by trapping and pulling the beads attached to the ‘target’ DNA molecules.

Single Molecule Isolation within Microfluidic Channels

A microfluidic device in accordance with one embodiment of the invention permitted easy identification of which DNA/bead complexes are immobilized. The efficiency of hybridization within microfluidic flows was lower than in the static case as expected, facilitating single molecule isolation at multiple dispersed locations on the substrate. The number of single molecules isolated within a microfluidic channel of 100 μm width and 1 cm length was approximately 10-20 molecules.

Single Molecule Release within Microfluidic Channels by Electrical Heating

After visualization of single molecule immobilization, single molecule release was achieved by heating the chip. Heating was performed using a thin film resistive heater located underneath the chip and controlling the current passing through the resistive heater. When the local temperature at the binding position on the substrate exceeds the melting temperature of the hybridized DNA molecule, the hybridized DNA molecule denatures and is released from the substrate.

For the DNA sequences that were chosen, the theoretical release temperature was 48.9° C. The releases of various single DNA molecules isolated were observed at substrate temperatures ranging from 46° C. to 53° C. This range was observed for single molecules released from the same microfluidic channel, as well as from multiple channels.

### Example 2

Substrate Modification

A glass surface is treated with alkaline solution (NaOH, 1N) to expose hydroxyl groups. The hydroxylated surface is subsequently treated with an aldehyde-containing silane reagent (10 millimolar in 95% ethanol) to provide an aldehyde-activated substrate. After washing with ethanol three times, and deionized water three times, the aldehyde-activated substrate is coated with a solution containing avidin and BSA (bovine serum albumin) in certain molar ratio: 1:10 or 1:1000, etc. The aldehydes react readily with primary amines on the proteins to form Schiff's base linkages between the aldehydes and the proteins, i.e., to covalently attach the proteins to the aldehyde-activated substrate surface.

A poly-(dT)30 oligonucleotide can be obtained commercially (Qiagen-Operon). The 5′ end is labeled with a biotin moiety. The oligonucleotide is allowed to bind to the coated glass surface, followed by washing/cleaning to remove free (non-attached) oligonucleotide molecules with 1×PBS.

Agent Preparation

Streptavidin coated micro-sphere (fluorescent) of 1 um can be purchased from a commercial source (Polysciences Inc.)

Target Molecule Preparation

A DNA sample is digested with two different restriction enzymes to create DNA fragments having two different ends (e.g., 10 micrograms of yeast DNA is digested in 100 microliters of 1× restriction enzyme digestion buffer (New England Biolabs), containing 50 units of EcoR1 and 50 units of BamH1). About 10 nanograms of a 20 kbp DNA fragment are isolated from agarose gel by methods known by those of ordinary skill in the art. A hairpin-like oligonucleotide (cap-oligo) with a biotin moiety in the middle and a restriction enzyme site at its end is synthesized and ligated to the desired end (determined by the restriction enzyme). After ligation, the DNA has a closed end with a biotin and an open end.

50 microliters of an enzyme solution containing terminal transferase (20 units) and 10 micromolar dATP can be used to add a poly dA tail (20-50 nucleotides long) to the open end of the DNA. Other end modification methods can also be used, depending on the final application of the molecule.

Single DNA Molecule Isolation

The target DNA is added to the substrate, and the oligo-dA tail hybridizes to the poly-dT nucleotide attached to the substrate surface in a standard hybridization buffer (1×PBS) that maintains proper pH and salt concentrations. The substrate is cleaned with 1×PBS buffer to remove free target molecules. The immobilized target molecules agents are contacted with an agent, here streptavidin coated microspheres to localize the target DNA molecules. The immobilized microspheres can be located with a microscope. Immobilization occurs when streptavidin binds to the biotin moiety in the DNA.

The presence of single DNA attaching to a carrier can be confirmed by an optical trapping technique. For example, an optical tweezers can stretch a DNA molecule by moving the carrier in different directions. The maximum stretching range (about 15 um for lambda DNA) and force applied can be measured and used as indicators for the presence of single molecule because the presence of more than one molecules will result in smaller stretching range for a given force.

To isolate a particular DNA molecule, a laser beam can be applied to the position where there is an immobilized microsphere, the local heating effect generated by the laser can denature the poly-dA and poly-dT hybrid, and thus release the DNA molecule (which is still attached to the microsphere) from the substrate surface. The DNA molecule is isolated by transporting the microsphere to a desired location using an optical tweezers.

### Example 3

Target Molecule Preparation

The ends of target DNA molecules are labeled with an appropriate functional group (for example, by using enzymes such as Klenow fragment and biotin-labeled nucleotides, if using streptavidin-coated beads) such that the labeled DNA molecules can bind to the surface of the beads. Labeling typically is performed on a dsDNA molecule, and then the strands are separated. A linker can be ligated to the 5′ end of the top strand of the double-stranded DNA and a poly dA tail can be added to the 3′ end of the same strand. Single stranded molecules having both ends modified can be obtained after denaturing the DNA.

Alternatively, the single-stranded DNA could be labeled at the 3′ (by polymerization or terminal transferase reaction) end or the 5′ end (by ligation). The 3′ and the 5′ ends can be labeled differently, for example, one end with digoxiginin and the other with biotin.

Formation of Agent-Polymeric Molecule Complexes

The end-labeled single stranded DNA molecules are mixed with surface-functionalized beads (e.g., microspheres coated with streptavidin) such that the DNA strands are in excess (i.e., there is more than one DNA molecule per bead). The end-labeled single-stranded DNA binds to the coated surface of the microspheres.

Substrate Modification

The surface of a substrate is functionalized with appropriate DNA oligonucleotides so that the attached oligomers can hybridize with at least a portion of the target, single-stranded DNA molecule of interest.

The density of the oligomers attached to the substrate surface is controlled by first contacting the substrate with different ratios of avidin and bovine serum albumin (BSA), e.g., 1:1000. The biotin-labeled oligomers are subsequently attached to the avidin.

Immobilization of Agent-Polymeric Complex to Substrate

The oligomer modified surface is contacted with the DNA-bead complex solution (e.g., by flowing the solution over the substrate). At least one of the DNA single-stranded molecules on the bead hybridizes with the oligomer attached to the substrate surface.

Removal of Polymeric Molecules on the Agent-Polymeric Molecules which are Not Immobilized to the Surface

The substrate surface is contacted with an enzyme solution (e.g., by flowing, etc.) to selectively deconstruct the single stranded DNA molecules on the surface of the bead while leaving the bead attached to the surface. In this case, an enzyme solution comprising exonuclease can be used. The enzyme solution should be 5′ or 3′ specific depending on the labeling of the target and the capture DNA (5′ specific if the single strand target DNA is labeled at the 3′ end and vice versa).

Single Molecule Release

The substrate surface is contacted with a solution (e.g., by flowing, etc.) to denature the hybridization between the oligomer attached to the substrate surface and the single-stranded DNA molecule of interest, which is attached to the bead, thereby causing the beads (and the target molecule of interest) to be released. Local heating of the surface can be performed such that the temperature exceeds the melting temperature of the hybridization to release the beads.

### Example 4

Target Molecule Preparation

For Labeled DNA:

A DNA sample is digested with two different restriction enzymes to create DNA fragments having two different ends. For example, 10 micrograms of yeast DNA is digested in 100 microliters of 1× restriction enzyme digestion buffer (New England Biolabs), containing 50 units of EcoR1 and 50 units of BamH1. About 10 nanograms of a 20 kbp DNA fragment are isolated from agarose gel by methods known in the art. A hairpin-like oligonucleotide (cap-oligo) with a biotin moiety in the middle and a restriction enzyme site at its end is synthesized. The cap oligo is ligated to the desired end of the target molecule (determined by the restriction enzyme, for example EcoR1). After ligation, the target DNA has a closed end with a biotin and an open end.

For Tailed DNA:

Terminal transferase can be used to add a poly dA tail (20-50 nucleotides long) to the ends of a DNA molecule. For example, 50 microliters of an enzyme solution containing 20 units of terminal transferase and 10 micromolar dATP can be used to add a poly dA tail between about 20 and about 50 nucleotides long.

Formation of Agent-Polymeric Molecule Complexes

For labeled DNA:

Streptavidin coated microspheres (fluorescent) can be obtained from a commercial source (Polysciences Inc). About 1 microgram of biotin-labeled DNA molecules is mixed with the microspheres (carriers) in a 1 molecule to 1 agent ratio in a binding buffer (1×PBS plus 0.1% Tween-20). The unbound DNA molecules are removed using centrifugation at 14,000×g for 10 min. The pellet is resuspended in the binding buffer (1×PBS plus 0.1% Tween-20) and the washing procedure (resuspending the bead-DNA complexes in binding buffer and centrifugation) is repeated two more times. Finally, the agent-polymeric molecule complexes (here, bead-DNA complexes) are resuspended in 50 microliters of the same binding buffer.

For Tailed DNA:

Streptavidin coated microspheres (fluorescent) can be obtained from a commercial source (Polysciences Inc). Mix biotin-labeled oligomer dT (1 micromolar in 1×PBS, plus 0.1% Tween-20) with the micro-spheres (carriers), and remove unbound oligonucleotides by centrifugation and washing, as described above. The tailed target DNA molecules can hybridize to the oligomer dT (on the agents) in a 1 molecule to 1 agent ratio in 50 microliters of the same buffer. The unbound DNA molecules are removed using centrifugation, as described above.

Single DNA Molecule Isolation According to Carrier Mobility

The carrier-DNA complexes are introduced to the sample well of a microfluidic device in accordance with an embodiment of the invention. A voltage is applied to separate the carriers along the length of the microchannel pathway. Agents having a single bound polymeric molecule can be isolated based on a predicted mobility corresponding to single DNA molecule attachment by directing the carrier to a collection chamber/channel using an additional applied electrical field and/or fluidic pressure/vacuum.

1. A method for isolating a single polymeric molecule comprising:
(a) contacting polymeric molecules with agents under conditions that permit formation of agent-polymeric molecule complexes, said polymeric molecules immobilized at binding positions on a substrate, said agents comprising a binding partner having binding affinity for a label of the polymeric molecules, and said binding positions separated by at least about two times the length of the polymeric molecules; and,
(b) releasing at least one of the agent-polymeric molecule complexes from the substrate to isolate a single polymeric molecule.

2. The method of claim 1, wherein the agents are microsphere beads having a coating comprising the binding partner.

3. The method of claim 1, further comprising washing the substrate to remove the agents which have not formed the agent-polymeric molecule complexes.

4. The method of claim 1, wherein the substrate is selected from the group consisting of gold substrates, aluminum substrates, glass substrates, silicon substrates, and polymeric substrates.

5. The method of claim 1, wherein the label is biotin and the binding partner is selected from the group consisting of avidin and streptavidin.

6. The method of claim 1, wherein the label is an antigen and the binding partner is an antibody for the antigen.

7. The method of claim 6, wherein the label is digoxigenin and the binding partner is anti-digoxigenin antibody.

8. The method of claim 1, wherein the polymeric molecule is a nucleic acid.

9. The method of claim 8, wherein the nucleic acid is a double stranded nucleic acid.

10. The method of claim 8, wherein the nucleic acid is deoxyribonucleic acid.

11. The method of claim 8, wherein the nucleic acid is ribonucleic acid.

12. The method of claim 1, wherein the polymeric molecules comprise a first oligonucleotide, and the binding positions comprise a second oligonucleotide, which is complementary to at least a portion of the first oligonucleotide.

13. The method of claim 12, wherein the polymeric molecules are immobilized by hybridizing the first oligonucleotide to the second oligonucleotide.

14. The method of claim 1, wherein the releasing is selected from the group consisting of heating, adding a pH adjusting compound, and adding a disrupting agent.

15. The method of claim 2, further comprising transporting the at least one agent-polymeric molecule complex.

16. A method for isolating a single polymeric molecule comprising:
(a) providing an agent-polymeric molecule complex having only one bound polymeric molecule;
(b) immobilizing the agent-polymeric molecule complex to a substrate at a position having a binding position that interacts with at least a portion of the polymeric molecule; and,
(c) releasing the agent-polymeric molecule complex to isolate a single polymeric molecule.

17. The method of claim 16, wherein the agent is a microsphere bead having a coating comprising a binding partner having binding affinity for a label of the polymeric molecule.

18. The method of claim 16, wherein the substrate is selected from the group consisting of gold substrates, aluminum substrates, glass substrates, silicon substrates, and polymeric substrates.

19. The method of claim 17, wherein the label is biotin and the binding partner is selected from the group consisting of avidin and streptavidin.

20. The method of claim 17, wherein the label is an antigen and the binding partner is an antibody for the antigen.

21. The method of claim 20, wherein the label is digoxigenin and the binding partner is anti-digoxigenin antibody.

22. The method of claim 16, wherein the polymeric molecule is a nucleic acid.

23. The method of claim 22, wherein the nucleic acid is a double stranded nucleic acid.

24. The method of claim 22, wherein the nucleic acid is deoxyribonucleic acid.

25. The method of claim 22, wherein the nucleic acid is ribonucleic acid.

26. The method of claim 16, wherein the polymeric molecule comprises a first oligonucleotide, and the binding position comprises a second oligonucleotide, which is complementary to at least a portion of the first oligonucleotide.

27. The method of claim 26, wherein the immobilizing comprises hybridization of the first oligonucleotide to the second oligonucleotide.

28. The method of claim 16, wherein the releasing is selected from the group consisting of heating, adding a pH adjusting compound, and adding a disrupting agent.

29. The method of claim 17, further comprising transporting the agent-polymeric molecule complex.

30. The method of claim 16, wherein the providing comprises removing the polymeric molecules on the agent-polymeric molecule complex that are not immobilized to the substrate.

31. A method for isolating a single polymeric molecule comprising:
(a) introducing a mixture comprising agent-polymeric molecule complexes into an applied electric field, said mixture including agent-polymeric molecule complexes having varying numbers of polymeric molecules bound to the agents; and,
(b) separating the agent-polymeric molecule complexes having only one bound polymeric molecule from the mixture based on mobility to isolate a single polymeric molecule.

32. The method of claim 31, wherein the agents are microsphere beads having a coating comprising a binding partner having binding affinity for a label of the polymeric molecule.

33. The method of claim 32, wherein the label is biotin and the binding partner is selected from the group consisting of avidin and streptavidin.

34. The method of claim 32, wherein the label is an antigen and the binding partner is an antibody for the antigen.

35. The method of claim 34, wherein the label is digoxigenin and the binding partner is anti-digoxigenin antibody.

36. The method of claim 31, wherein the polymeric molecule is a nucleic acid.

37. The method of claim 36, wherein the nucleic acid is a double stranded nucleic acid.

38. The method of claim 36, wherein the nucleic acid is deoxyribonucleic acid.

39. The method of claim 36, wherein the nucleic acid is ribonucleic acid.

40. The method of claim 31, further comprising determining the mobility of an agent-polymeric molecule complex having only one bound polymeric molecule under the applied electric field.

41. A microfluidic device comprising:
(a) a micromold comprising a chemically inert material and having a top surface, a bottom surface, a sample inlet, a sample outlet, and a microchannel pathway defined between the sample inlet and the sample outlet;
(b) a substrate adhered to the bottom surface, the substrate having binding positions for immobilizing polymeric molecules, said binding positions separated by at least about two times the length of the polymeric molecules; and,
(c) a heating element adapted to heat the substrate.

42. The microfluidic device of claim 41, wherein the micromold comprises a silicone material.

43. The microfluidic device of claim 41, wherein the microchannel has a width between about 10 microns and about 200 microns.

44. The microfluidic device of claim 41, wherein the microchannel has a length between about 0.25 centimeters and about five centimeters.

45. The microfluidic device of claim 41, wherein the binding positions comprise a polymeric molecule.

46. The microfluidic device of claim 45, wherein the polymeric molecule comprises a thiol-modified oligonucleotide.

47. The microfluidic device of claim 45, wherein the polymeric molecule comprises a labeled oligonucleotide.

48. The microfluidic device of claim 45, wherein the heating element comprises a thin-film resistive heater.

49. The microfluidic device of claim 48, wherein the heating element is the substrate.

50. The microfluidic device of claim 45, further comprising a passivation layer between the substrate and the heating element.

51. The microfluidic device of claim 50, wherein a first pattern formed by the resistive heater is different from a second pattern formed by the substrate.

52. The microfluidic device of claim 51, wherein the first pattern and the second pattern intersect at locations, thereby providing individually addressable binding positions.

53. A microfluidic device comprising:
(a) a micromold comprising a chemically inert material and having a sample well, a first end, a second end, and a microchannel pathway defined between the first end and the second end; and,
(b) a first electrode disposed proximate to the first end and a second electrode disposed proximate to the second end.

54. The microfluidic device of claim 53, further comprising a collection chamber having a third end and a fourth, collection end, the collection chamber being substantially transverse to the microchannel.

55. The microfluidic device of claim 54, further comprising a third electrode disposed proximate to the third end and a fourth electrode disposed proximate to the fourth, collection end.

56. The microfluidic device of claim 53, further comprising a switching circuit between the first electrode and the second electrode.

57. The microfluidic device of claim 56, further comprising a power supply operatively connected to the switching circuit.
